(19) United States
(12) Patent Application Publication
(10) Pub. No.: US 2025/0345260 A1
(43) Pub. Date: Nov. 13, 2025

(54) COSMETIC PRODUCT FOR TEMPORARILY SHAPING KERATIN-CONTAINING FIBERS

(71) Applicant: HENKEL AG & CO. KGAA, Düsseldorf (DE)

(72) Inventors: Thorsten KNAPPE, Schenefeld (DE); Pamela KAFTAN, Hamburg (DE); Alexandra GROSSDORF, Schenefeld (DE)

(21) Appl. No.: 18/871,415

(22) PCT Filed: Feb. 23, 2023
(86) PCT No.: PCT/EP2023/054563

§ 371 Date: Dec. 3, 2024

(30) Foreign Application Priority Data
Jun. 15, 2022 (DE) 10 2022 115 088.7

(51) Int. Cl. A61K 8/73; A61K 8/04; A61K 8/19; A61Q 5/06

(52) U.S. Cl. CPC A61K 8/731; A61K 8/046; A61K 8/19; A61K 8/732; A61K 8/737; A61Q 5/06

(57) ABSTRACT

A cosmetic product is in the form of a foam and is for temporarily shaping keratin-containing fibers. The cosmetic product includes at least one non-fully synthetic film-forming agent and at least nitrogen as the propellant. Use the cosmetic product for temporarily shaping keratin-containing fibers, and a method for temporarily shaping keratin-containing fibers using the cosmetic product are also described.

The present invention relates to a cosmetic product which is used in the form of a foam, for temporarily shaping keratin-containing fibers, and comprises at least one non-fully synthetic film-forming agent and at least nitrogen as a propellant, and to the use of the product for temporarily shaping keratin-containing fibers, and to a method for temporarily shaping keratin-containing fibers using the cosmetic product according to the invention.

Cosmetic products which permanently or temporarily shape hair play an important role in cosmetics. Temporary shaping, which is intended to provide a good hold without impairing the healthy appearance of the hair, such as its shine, can be achieved, for example, by hair sprays, hair waxes, hair gels, hair foams, hairdryers, etc.

The most important property of a product for temporarily shaping keratin-containing fibers is that of providing the treated fibers with the strongest and longest possible hold in the shape created. If the keratin-containing fibers are human hair, this is also referred to as strong hairstyle hold, or a high degree of hold of the cosmetic product. Hairstyle hold is substantially determined by the type and quantity of polymers used.

Numerous cosmetic products which provide temporary shaping are known from the prior art. Corresponding products usually contain synthetic polymers as shaping components. Said products often cause the hair to dry out.

For simple use and uniform working of the product into keratin-containing fibers, in particular into human hair, the product is preferably provided as a foam. In order to produce the foam (an aerosol), a propellant is required. Conventional propellants are, for example, alkanes having 3 to 5 carbon atoms, such as propane, n-butane, isobutane, n-pentane, and isopentane, and mixtures thereof. These have the disadvantage of not being regarded as sustainable.

Sustainability is becoming increasingly important among consumers, including with regard to cosmetic products. This relates to all components, i.e. also to film-forming agents and propellants. There is therefore a need for a cosmetic product in which both the propellant and the film-forming agent are more sustainable than conventionally used components. However, the styling properties should not be adversely affected.

Surprisingly, it has been found that non-fully synthetic film-forming agents, in combination with nitrogen as the propellant, also allow temporary shaping of hair. In a first embodiment, the problem addressed by the present invention is therefore solved by a cosmetic product for temporarily shaping keratin-containing fibers, in particular human hair, which is applied in the form of a foam, comprising at least one non-fully synthetic film-forming agent, and at least nitrogen as a propellant.

Surprisingly, it has been found that cosmetic products which are not inferior to conventional products in respect of styling properties can also be obtained using non-fully synthetic film-forming agents. In addition, previous foams were produced using short-chain hydrocarbons as the propellant. The assumption was that no stable foams can be obtained using other propellants. However, it has been shown that cosmetic products in the form of a foam can be obtained using nitrogen, even when using non-fully synthetic film-forming agents.

According to the invention, the cosmetic product is applied in the form of a foam, and therefore contains at least nitrogen as the propellant. The amount of propellant is dependent on the type and size of the selected container in which the cosmetic product is offered and used. The amount of propellant in the cosmetic product is therefore selected such that a pressure of 5 bar to 15 bar, in particular of 7 bar to 12 bar, is produced in the container containing the cosmetic product. Such a pressure is usually selected in order to produce a good foam and to empty a container as completely as possible. Higher pressures can be used by selecting packagings with correspondingly designed pressure resistance, but are not common due to the required higher material use.

According to the invention, the cosmetic product contains nitrogen as the propellant. However, also according to the invention, the cosmetic product additionally contains further propellants. These further propellants are in particular selected from N₂O, dimethyl ether, CO₂, air, alkanes having 3 to 5 carbon atoms, such as propane, n-butane, isobutane, n-pentane, and isopentane, and mixtures thereof. If the cosmetic product contains, in addition to nitrogen, one, two or more further propellants, the total pressure in the container in which the cosmetic product is used is in the range of 5 bar to 15 bar, in particular 7 bar to 12 bar.

The cosmetic product therefore preferably contains the at least one propellant in amounts of from 0.1 to 20 wt. %, based on the total product. Amounts of 0.2 to 10 wt. %, in particular 0.3 to 5 wt. %, are particularly preferred, the amount relating to the total weight of the cosmetic product. If the cosmetic product contains more than one propellant, this is the amount of all propellants contained.

According to the invention, the cosmetic product furthermore comprises at least one non-fully synthetic film-forming agent. Film-forming agents are understood to be polymers which leave behind a continuous film on the skin, hair or nails upon drying. Film-forming agents of this kind can be used in a wide variety of cosmetic products, such as face masks, make-up, hair setting products, hairsprays, hair gels, hair waxes, hair masques, shampoos or nail polishes. Preference is given to polymers which possess a sufficient solubility in water, alcohol or water/alcohol mixtures. Corresponding solutions can thus be produced which can be used or further processed in a simple manner.

Within the scope of the present invention, a film-forming agent is understood to mean, in particular, polymers which, when used in 0.05 to 20 wt. % (based on the total weight of the cosmetic product) of aqueous, alcoholic, or aqueous-alcoholic solution, are able to be deposited on the hair in the form of a transparent polymer film which makes it possible to temporarily bond hair fibers to one another.

According to the invention, the cosmetic product contains one or more non-fully synthetically produced film-forming agents (non-fully synthetic film-forming agents).

In principle, film-forming agents are known from the prior art. Usually, exclusively fully synthetically produced film-forming agents (fully synthetic film-forming agents) are used in cosmetic products. Within the scope of the present invention, a film-forming agent is to be considered fully synthetic if all of the monomers which make up the film-forming agent are conventionally produced synthetically. The film-forming agent is to be regarded as fully synthetically produced even if a monomer was obtained from biogas or other renewable sources, for example.

In contrast to this, the non-fully synthetically produced film-forming agents are those which are based on a naturally occurring substance. According to the invention, said naturally occurring substance (natural substance) can be chemically modified. These film-forming agents based on natural substances are referred to, in the present case, as non-fully synthetically produced or non-fully synthetic film-forming agents.

Non-fully synthetically produced film-forming agents, within the meaning of the present invention, are polymers or copolymers which are based on a natural substance. According to the invention, these natural substances can still be chemically modified.

Suitable natural substances are, for example, saccharides (mono-, di-and polysaccharides) or biopolymers (polymers synthesized in the cell of a living being).

Suitable saccharides are, for example, glucose, fructose, sucrose, lactose, maltose, cellulose or starch. Plant rubbers (polysaccharides of vegetable origin), such as guar gum (guar), agar, carrageenan, acacia senegal gum or others are also suitable in the present case. A suitable biopolymer is chitosan. These compounds, in particular chemically modified, can be used as film-forming agents, alone or in mixtures, in the cosmetic product according to the invention.

Suitable cellulose derivatives are in particular hydroxypropyl cellulose, having a molecular weight of 30,000 to 50,000 g/mol, cationic cellulose derivatives, in particular those which result from a reaction of hydroxyethyl cellulose with a dimethyl diallylammonium reactant (in particular dimethyl diallylammonium chloride).

Also suitable are polysaccharides, which are in particular obtained from starch, for example the polysaccharide having the INCI name pullulan. Also suitable are, for example, potato starch, or a starch modified by non-ionic propylene oxide, as described in DE 10 2017 222 854 A1 or DE 10 2018 218 270 A1.

Starch is a reserve carbohydrate which is stored by many plants in the form of starch grains (granules) usually 1 to 200 μm in size in different plant parts, for example in tubers or roots, cereal seeds, fruits and in the pith. A useable, non-ionic, propylene oxide-modified starch can be derived from starch from potatoes, corn, rice, peas, acorns, chestnuts, barley, wheat, bananas, sago, millet, sorghum, oats, barley, rye, beans, sweet potato, arrowroot or manioc. Particularly pronounced advantageous effects are achieved by non-ionic, propylene oxide-modified tapioca starch or non-ionic, propylene oxide-modified potato starch, or by mixtures of both aforementioned starches. The cosmetic product very particularly preferably contains at least one non-ionic, propylene oxide-modified potato starch. A non-ionic propylene oxide-modified starch is thus understood to mean a reaction product of a starch and propylene oxide. Pea starch or a modified pea starch can also preferably be used, the modified pea starch preferably being a non-ionic, propylene oxide-modified pea starch.

Furthermore, guar hydroxypropyltrimonium chloride can be used according to the invention as a non-fully synthetic film-forming agent, if it has film-forming properties.

Chitosans are biopolymers and belong to the group of hydrocolloids. Considered chemically, these are partially deacetylated chitins of different molecular weight. For the preparation of the chitosans, chitin is used, preferably the shell residues of crustaceans, which are available in large amounts as cheap raw materials. The chitin is usually first deprotonated by the addition of bases, demineralized by the addition of mineral acids, and finally deacetylated by the addition of strong bases. Alternatively, the deacetylation can also take place enzymatically. The molecular weights can be distributed over a wide spectrum. Types are preferably used which have an average molecular weight (weight average) of from 800,000 to 1,200,000 daltons, a Brookfield viscosity (1 wt % in glycolic acid) below 5000 mPas, a degree of deacetylation in the range of from 80 to 88%, and an ash content of less than 0.3 wt %.

In addition to the chitosans, cationically derivatized chitosans (such as quaternizing products) or alkoxylated chitosans can also be used.

Suitable chitosans are freely available commercially, for example under the trade names Hydagen® CMF (1 wt. % of active substance in aqueous solution with 0.4 wt. % of glycolic acid, molecular weight 500,000 to 5,000,000 g/mol; Cognis), Hydamer® HCMF (80% deacetylated chitosan, molecular weight 50,000 to 1000000 g/mol, Chitinor, formerly Cognis), Kytamer® PC (about 80 wt. % of active substance chitosan pyrrolidone carboxylate (INCI name: Chitosan PCA), Amerchol), Chitolam® NB/101 and Chitosan 90/100/A1 (about 90% deacetylated chitosan, BioLog Heppe).

Very particularly preferably, the cosmetic products can contain neutralization products of chitosan, having at least one acid as chitosan derivative(s). In the context of the present invention, the neutralization product is any product which is produced by conversion of chitosan using an organic carboxylic acid, i.e. the neutralization product can be prepared separately before the preparation of the cosmetic product, or the neutralization product can be formed in situ during the production of the cosmetic product.

According to a preferred embodiment of the present invention, the organic carboxylic acid is to be selected from formic acid, acetic acid, citric acid, lactic acid, pyrrolidone carboxylic acid, tartaric acid, glycolic acid, nicotinic acid, hydroxyisobutyric acid, hydroxyisovaleric acid, or mixtures of these acids, in particular lactic acid. This neutralization product can be prepared, for example, in an aqueous medium, by adding chitosan and the corresponding organic carboxylic acid, or a portion of the corresponding organic carboxylic acid.

According to a preferred embodiment of the present invention, the weight ratio of chitosan to the organic acid is between 2:1 and 1:10, preferably between 1:1 and 1:6, even more preferably between 1:2 and 1:4. In this more preferred embodiment, the total weight of the chitosan used is based on the total weight of the organic acid in the cosmetic product. A particularly preferred organic acid is lactic acid. Lactic acid is a solubility enhancer. Used in the above weight ratios, it achieves particularly advantageous styling effects and gives the hair an exceptional hairstyle hold.

The cosmetic product according to the invention contains at least one non-fully synthetically produced film-forming agent. According to the invention, it can also have mixtures of two, three or more non-fully synthetically produced film-forming agents. The cosmetic product, according to the invention, can furthermore contain at least one fully synthetically produced film-forming agent. According to the invention, the cosmetic product therefore comprises one, two, three or more different non-fully synthetically produced film-forming agents. Additionally, it may have one, two, three or more non-fully synthetically produced film-forming agents.

In a preferred embodiment, the cosmetic product according to the invention is substantially free of fully synthetically produced film-forming agents. Surprisingly, it has been found that a cosmetic product which is substantially free of fully synthetic film-forming agents is not necessarily associated with losses in styling properties. In the context of the present invention, the cosmetic product can comprise up to 5 wt. %, in particular up to 3 wt. %, preferably up to 2 wt. %, more preferably 1 wt. % or less of fully synthetic film-forming agents, based on the total weight of the product, in order to be “substantially” free of fully synthetic film-forming agents. Preferably, the cosmetic product can also be completely free of fully synthetic film-forming agents, i.e. the cosmetic product comprises 0 wt. % of fully synthetic film-forming agents.

Within the scope of the present invention, a film-forming agent is to be considered fully synthetically produced if all of the monomers which make up the film-forming agent are conventionally produced synthetically. If at least one monomer is of natural origin, the film-forming agents are to be regarded as non-fully synthetically produced. Non-fully synthetic film-forming agents thus also include film-forming agents which are completely of natural origin.

Examples of film-forming agents which are usually produced fully synthetically comprise in particular copolymers based on acrylate or methacrylate, in particular those which comprise acrylic acid, methacrylic acid, alkyl(meth)acrylate, hydroxyalkyl(meth)acrylate and/or maleic anhydride as a monomer component, and copolymers which comprise N-vinylpyrrolidone, alkyl vinyl ethers with alkyl groups comprising 1 to 18 carbon atoms and/or vinyl esters of carboxylic acids having 2 to 18 carbon atoms as monomer components, in particular polyvinylpyrrolidone/vinyl acetate copolymers; and also polyvinylpyrrolidone; wherein copolymers are understood to be polymers consisting of two or more different monomers, and all alkyl groups of the monomers mentioned can also be branched alkyl groups and/or alkyl groups substituted with functional groups. Corresponding fully synthetic film-forming agents are well known to a person skilled in the art.

The product according to the invention contains film-forming agents preferably in a proportion of from 0.05 wt. % to 30 wt. %, in particular from 0.10 wt. % to 20 wt. %, preferably from 0.15 wt. % to 15 wt. %, based on the total weight of the cosmetic product. The specified amounts are in each case based on all film-forming agents present when mixtures of a plurality of film-forming agents are contained.

Keratin-containing fibers are understood in the context of the present invention to mean fur, wool, feathers, and hair, in particular human hair. This is particularly preferably understood to be textured human hair, i.e. curly or wavy hair. In particular in the case of textured hair, the desire for a high proportion of care ingredients is high, since the hair quickly becomes brittle.

According to a preferred embodiment of the present invention, the cosmetic product contains a care polymer in small amounts, which polymer is particularly preferably a cationic homopolymer. Cationic polymers are understood to be polymers which comprise a group, in the main chain and/or side chain, that can be “temporarily” or “permanently” cationic. According to the invention, the term “permanently” cationic refers to polymers that comprise a cationic group irrespective of the pH of the product. These are generally polymers that contain a quaternary nitrogen atom, for example in the form of an ammonium group. Preferred cationic groups are quaternary ammonium groups.

In particular homopolymers in which the quaternary ammonium groups are attached to a homopolymer main chain constructed from acrylamide via a C₁₋₄ alkylene group, the three alkyl groups other than the C₁₋₄ alkylene group of the ammonium group being chosen independently of one another from C₁₋₁₀ alkyl groups, have proven to be particularly suitable.

All possible physiologically acceptable anions, such as acetate, sulphate, chloride, bromide, and/or phosphate, may be used to compensate for the positive polymer charge.

The product can also contain as a care component, for example, at least one protein hydrolyzate and/or one of the derivatives thereof. Protein hydrolyzates are product mixtures that are obtained by acid-, base-, or enzyme-catalyzed degradation of proteins. The term protein hydrolyzates is also understood to mean total hydrolyzates, as well as individual amino acids and derivatives thereof, and mixtures of different amino acids. The molar weight of the usable protein hydrolyzates is between 75, the molar weight of glycine, and 200,000; the molar weight preferably being 75 to 50,000 daltons, and very particularly preferably 75 to 20,000 daltons.

Likewise suitable care agents, according to the invention, are in particular quaternary ammonium derivatives, in particular BTAC or CTAC.

The product according to the invention can furthermore contain at least one vitamin, a provitamin, a vitamin precursor, and/or one of the derivatives thereof as a care component. In this case, the vitamins, provitamins, and vitamin precursors that are usually assigned to the groups A, B, C, E, F, and H are preferred. A particularly preferred care component is D-panthenol.

The advantage of said care components is that they originate largely from natural sources, and are thus raw materials that are energy-saving and resource-saving. According to the preferred embodiment, the cosmetic product, based on the total weight, can contain 0.0005 to 3 wt. %, preferably 0.001 to 2 wt. %, and particularly preferably 0.05 to 1 wt. % of the sum of care oils and care components.

According to a further preferred embodiment, the cosmetic product can additionally contain an emulsifier. Emulsifiers lead to the formation, at the phase interface, of water-resistant or oil-resistant adsorption layers, which prevent the dispersed droplets from coalescing and thus stabilize the emulsion. Like surfactants, emulsifiers are therefore formed from a hydrophobic and a hydrophilic molecule part. Hydrophilic emulsifiers preferably form O/W-emulsions (oil-in-water emulsions) and hydrophilic emulsifiers preferably form W/O-emulsions (water-in-oil emulsions). Particularly preferred emulsifiers are alkoxylated fatty alcohols. The particularly preferred emulsifiers offer the advantage of being able to be obtained from natural, non-fully synthetic raw material sources.

Accordingly, in a preferred embodiment, the cosmetic product further contains at least one alkoxylated fatty alcohol.

An “alkoxylated fatty alcohol” is any fatty alcohol with a carbon chain of C₅ or more, which further comprises at least one alkoxy group. The fatty alcohol can, for example, be selected from C₉-C₁₁ fatty alcohols, C₁₂-C₁₃ fatty alcohols, C₁₂-C₁₅ fatty alcohols, C₁₂-C₁₆ fatty alcohols, C₁₄-C₁₅ fatty alcohols, arachidyl alcohol, behenyl alcohol, caprylic alcohol, cetearyl alcohol, cetyl alcohol, coconut alcohol, decyl alcohol, (hydrogenated) tallow alcohol, lauryl alcohol, myristyl alcohol, oleyl alcohol, palm alcohol, palm kernel alcohol, stearyl alcohol, and tridecyl alcohol.

The alkoxy group can in particular comprise ethoxy and/or propoxy and/or butoxy groups. The alkoxylated fatty alcohol is particularly preferably an ethoxylated fatty alcohol.

The at least one alkoxy group of the “alkoxylated fatty alcohol” can be derived, for example, from an alkoxylation reaction with alkylene oxide, in particular ethylene oxide and/or propylene oxide.

It is preferred that the at least one alkoxylated fatty alcohol is selected from the group consisting of ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-15, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, ceteareth-33, ceteareth-34, ceteareth-40, ceteareth-50, ceteareth-55, ceteareth-60, ceteareth-80, ceteareth-100, laureth-1, laureth-2, laureth-3, laureth-4, laureth-5, laureth-6, laureth-7, laureth-8, laureth-9, laureth-10, laureth-11, laureth-12, laureth-13, laureth-14, laureth-15, laureth-16, laureth-20, laureth-23, laureth-25, laureth-30, laureth-40, deceth-3, deceth-5, oleth-5, oleth-30, steareth-2, steareth-4, steareth-6, steareth-7, steareth-10, steareth-11, steareth-13, steareth-14, steareth-15, steareth-20, steareth-21, steareth-25, steareth-27, steareth-30, steareth-40, steareth-50, steareth-100 and mixtures thereof.

The designation ceteareth-2 is in this case, for example, a C₁₆-C₁₈ fatty alcohol having an average of 2 ethylene oxide units per molecule.

In a very particularly preferred embodiment of the cosmetic product, the at least one alkoxylated fatty alcohol comprises cetearyl alcohol and/or ceteareth-20, In particular cetearyl alcohol and ceteareth-20.

The amount of the at least one alkoxylated fatty alcohol is preferably 1 to 15 wt. % and more preferably 2 to 8 wt. %, in each case based on the total amount of the cosmetic product.

According to the invention, the cosmetic products can additionally have at least one surfactant. In general, non-ionic, anionic, cationic and ampholytic surfactants can be contained in cosmetic products. The group of the ampholytic or amphoteric surfactants comprises zwitterionic surfactants and ampholytes. It has been found to be particularly advantageous if the cosmetic products additionally contain at least one cationic surfactant for increasing hair-care properties.

It is preferably possible to use cationic surfactants of the following types: quaternary ammonium compounds, esterquats, and amidoamines. Preferred quaternary ammonium compounds are ammonium halides, in particular chlorides and bromides, such as alkyl trimethylammonium chlorides, dialkyl dimethylammonium chlorides, and trialkyl methylammonium chlorides. The long alkyl chains of these surfactants preferably have 10 to 308 carbon atoms, such as in, for example, cetyl trimethylammonium chloride, stearyl trimethylammonium chloride, distearyl dimethylammonium chloride, lauryl dimethylammonium chloride, lauryl dimethylbenzylammonium chloride, and tricetyl methylammonium chloride. According to a preferred embodiment of the present invention, alkyl trimethylammonium salts are used, preferably C₁₂-C₂₅ alkyl trimethylammonium salts, particularly preferably C₁₆-C₂₅ alkyl trimethylammonium chlorides, in particular cetrimonium chloride (CTAC) or behentrimonium chloride (BTAC).

In a preferred embodiment of the present invention, the cosmetic product contains 0.01 to 4 wt. %, particularly preferably 0.1 to 3 wt. %, and in particular 0.2 to 2 wt. %, of the cationic surfactant, the specified amounts relating to the total weight of the cosmetic product.

The applicability of the cosmetic product can be further increased by the use of small amounts of one or more polyhydric alcohols. Preferred cosmetic products contain one or more C₃ to C₁₂ alkane-1,2-diols, more preferably C₃ to C₁₀ alkane-1,2-diols. The cosmetic product particularly preferably contains propane-1,2-diol and/or caprylyl glycol. These particularly preferred diols improve the applicability, are so-to-speak nourishing, and are suitable for forming a stable emulsion with the particularly preferred emulsifier. Based on the total weight of the cosmetic product, in a preferred embodiment of the present invention, 0.01 to 1 wt. %, more preferably 0.02 to 0.8 wt. % of the alkane-1,2-diol, based in each case on the total weight of the cosmetic product, are contained in the cosmetic product.

In further preferred embodiments of the present invention, the cosmetic product further contains perfume and optionally contains further auxiliaries, such as means for adjusting the viscosity. These thickeners are preferably also non-synthetic, just like the film-forming agent.

The cosmetic product contains the ingredients described above in a cosmetic carrier. The cosmetic carrier is preferably an aqueous or aqueous-alcoholic carrier. The cosmetic product contains, based on its total weight, preferably at least 25 wt. %, more preferably at least 40 wt. %, and particularly preferably at least 60 wt. % of water. The cosmetic product particularly preferably comprises at least 65 wt. % of water, particularly preferably at least 70 wt. % of water or at least 75 wt. % of water.

Insofar as specified amounts are mentioned in the present application in wt. %, these refer to the active substance of the relevant substance based on the total weight of the product, unless stated otherwise.

The cosmetic products are preferably provided as foam. For this purpose, the cosmetic products are packaged in a dispensing device, which is either a compressed gas container (“aerosol container”) which is additionally filled with a propellant, or a non-aerosol container. The compressed gas containers, by means of which a product is dispensed via a valve by means of the internal gas pressure of the container, are referred to by definition as “aerosol containers”. Conversely to the aerosol definition, a container under standard pressure, by means of which a product is dispensed by a pumping or squeezing system by means of mechanical action, is defined as a “non-aerosol container”.

In a further embodiment, the problem addressed by the present invention is solved by the use of the cosmetic product as described above for temporarily shaping and/or setting the shape of keratin-containing fibers, in particular human hair.

In yet another embodiment, the problem addressed by present invention is solved by means of a method for temporarily shaping keratin-containing fibers, in particular human hair, characterized in that the cosmetic product is applied to the keratin-containing fibers as described above.

It has been found to be preferable that the keratin fibers are not rinsed after the action of the cosmetic product of the first subject matter of the invention, and said product is left on the fiber.

Features relating to preferred embodiments of the first embodiment, which are described above only in this regard, naturally also apply accordingly to the further embodiments as features of preferred embodiments.

The following examples are intended to explain the subject of the present invention, without limiting it in any manner.

### EXAMPLES

The following formulations were produced:

| | Ex. 1 | Ex. 2 | Ex. 3 | Ex. 4 | Ex. 5 | Ex. 6 |
|---|---|---|---|---|---|---|
| | wt. % | wt. % | wt. % | wt. % | wt. % | wt. % |
| | (active | (active | (active | (active | (active | (active |
| Ingredient | substance) | substance) | substance) | substance) | substance) | substance) |
| Non-fully | 4.6 | 8 | 1 | 1.96 | 1.96 | 1.9 |
| synthetic film- | | | | | | |
| forming agent | | | | | | |
| Fully synthetic | 1 | 0.5 | 1 | — | 1 | 1 |
| film-forming | | | | | | |
| agent | | | | | | |
| Care agent | 5 | 0.2 | 7 | 2.1 | 2.1 | — |
| Emulsifier | 1.1 | 1.2 | 1.0 | 1.5 | 1.1 | 1 |
| Surfactants | 2 | 5 | 0.5 | 1 | 1 | 1 |
| Fragrance | 0.15 | 0.2 | 0.10 | 0.15 | 0.15 | 0.15 |
| Auxiliaries | 0.5-1.5 | 0.5-1.5 | 0.5-1.5 | 0.5-1.5 | 0.5-1.5 | 0.5-1.5 |
| Water | up to 100 | up to 100 | up to 100 | up to 100 | up to 100 | up to 100 |
| (demineralized) | | | | | | |

Conventional pH adjusters, UV stabilizers, dyes, preservatives and thickeners were combined as auxiliaries.

The formulations were introduced into a conventional aerosol container. Nitrogen was exclusively used as the propellant. The amount of nitrogen was at most 2.30 wt. %, based on the total weight of the product. The pressure in the interior of the container was set to 10 bar to 12 bar.

It was possible to obtain a stable foam which was distributed uniformly in the hair. In formulations according to the invention, the hair received a natural shine and a strong but flexible hairstyle hold. In addition, the subjects had a positive hair feel.

1. A cosmetic product for temporarily shaping keratin-containing fibers, the cosmetic product comprising:
at least one non-fully synthetic film-forming agent; and
a propellant comprising nitrogen,
wherein the cosmetic product is in the form of a foam.

2. The cosmetic product of claim 1, wherein the at least one non-fully synthetic film-forming agent comprises a saccharide.

3. The cosmetic product of claim 2, wherein the non-fully synthetic film-forming agent further comprises a cellulose, a cellulose derivative, a starch, a modified starch, a pullulan, and/or guar hydroxypropyltrimonium chloride.

4. The cosmetic product of claim 1, further comprising a fully synthetic film-forming agent.

5. The cosmetic product of claim 1, wherein the cosmetic product is substantially free of fully synthetic film-forming agents.

6. The cosmetic product of claim 1, wherein the at least one non-fully synthetic film-forming agent is from 0.1 wt. % to 30 wt. % of the total weight of the cosmetic product.

7. The cosmetic product of claim 1, wherein the propellant furhter comprises at least one of N₂O, dimethyl ether, CO₂, air and alkanes having 3 to 5 carbon atoms, propane, n-butane, isobutane, n-pentane, or isopentane.

8. The cosmetic product of claim 1, wherein the amount of the propellant is sufficient to generate a pressure of 5 bar to 15 bar in a container containing the cosmetic product.

9. Use of the cosmetic product of claim 1 for temporarily shaping keratin-containing fibers.

10. A method for temporarily shaping keratin-containing fibers, the method comprising:
applying the cosmetic product of claim 1 to the keratin-containing fibers in the form of a foam.

11. The method of claim 10, wherein the cosmetic product is left on the keratin-containing fibers and is not rinsed out.

12. The cosmetic product of claim 1, wherein the keratin-containing fibers are human hair.

13. The cosmetic product of claim 2, wherein the saccharide comprises a monosaccharide, a disaccharide, a polysaccharide, and/or a biopolymer that is chemically modified.

14. The cosmetic product of claim 6, wherein the at least one non-fully synthetic film-forming agent is from 0.15 wt. % to 20 wt. % of the total weight of the cosmetic product.

15. The cosmetic product of claim 14, wherein the at least one non-fully synthetic film-forming agent is from 0.2 wt. % to 15 wt. % of the total weight of the cosmetic product.

16. The cosmetic product of claim 8, wherein the amount of the propellant is sufficient to generate a pressure of 7 bar to 12 bar in the container containing the cosmetic product.

17. The use of claim 9, wherein the keratin-containing fibers are human hair.

18. The method of claim 10, wherein the keratin-containing fibers are human hair.
